(19) United States
(12) Patent Application Publication
(10) Pub. No.: US 2025/0228699 A1
(43) Pub. Date: Jul. 17, 2025

(54) SNORING AND SLEEP APNEA PREVENTION SYSTEM USING SNORING AND SLEEP APNEA PREVENTION DEVICE

(71) Applicant: MD STAAGE INC., Seoul (KR)

(72) Inventors: Seong Taek KIM, Seoul (KR); Hee Jin KIM, Seoul (KR)

(21) Appl. No.: 18/844,435

(22) PCT Filed: Mar. 7, 2023
(86) PCT No.: PCT/KR2023/003063

§ 371 Date: Sep. 6, 2024

(30) Foreign Application Priority Data
Mar. 8, 2022 (KR) 10-2022-0029398

(51) Int. Cl. A61F 5/56; A61B 5/00

(52) U.S. Cl. CPC A61F 5/566; A61B 5/0002; A61B 5/4818; A61B 5/6826

(57) ABSTRACT

A snoring and sleep apnea preventer to prevent snoring and sleep apnea from occurring during a user's sleep comprises a sensor part mounted on a body part of the user and collecting sensing information to determine snoring and sleep apnea symptoms occurring in the user; an operating part mounted in the user's mouth to move the user's mandible in response to the sensing information; and a control part for controlling the operating part based on the sensing information sensed via the sensor part, and controlling the operating part in stages to control snoring and sleep apnea symptoms occurring in the user.

### TECHNICAL FIELD

The present invention relates to a snoring and sleep apnea prevention system using a snoring and sleep apnea preventer, and more particularly to a snoring and sleep apnea prevention system using a snoring and sleep apnea preventer that measures at least one of lung volume, body temperature, noise and vibration, and oxygen saturation through a sensor part to determine snoring and sleep apnea symptoms occurring during sleep of a user, and forms a preset routine to prevent snoring and sleep apnea symptoms of the user.

### BACKGROUND ART

During sleep, we do a variety of things without realizing it, which are commonly referred to as sleepwalking or sleep talking.

Snoring and sleep apnea, among other things, can be caused by a variety of factors, but it can also be caused by the lower jaw descending due to gravity and compressing the airway.

The problem with snoring and sleep apnea was that if it became severe enough, the person could suffocate due to decreased blood oxygenation.

That's why snoring and sleep apnea can be so severe that it's imperative to treat them quickly, especially since they can be fatal.

However, since snoring and sleep apnea are symptoms that occur during sleep, it is difficult to identify the exact intensity or event, and in order to properly identify them, it is necessary to directly observe the patient's sleep state, and in addition, even during treatment, it is difficult to precisely control and treat the patient because the patient cannot identify their own condition during sleep.

Therefore, various methods are being devised to determine the extent of a patient's snoring and sleep apnea, and to alleviate or prevent snoring and sleep apnea in patients, and methods are needed to do so.

### DISCLOSURE

### Technical Problem

The present invention is designed to solve the problems of the prior art described above, and aims to prevent snoring and sleep apnea by measuring at least one of lung volume, body temperature, noise and vibration, and oxygen saturation through a sensor part to determine snoring and sleep apnea symptoms that occur during sleep, and forming a preset routine based on the results to prevent snoring and sleep apnea symptoms of a user.

The tasks of the present invention are not limited to those mentioned above, and other tasks not mentioned will be apparent to those skilled in the art from the following description.

### Technical Solution

A snoring and sleep apnea preventer to prevent snoring and sleep apnea from occurring during a user's sleep, comprises A sensor part mounted on a body part of the user and collecting sensing information to determine snoring and sleep apnea symptoms occurring in the user: an operating part mounted in the user's mouth to move the user's mandible in response to the sensing information: and a control part for controlling the operating part based on the sensing information sensed via the sensor part, and controlling the operating part in stages to control snoring and sleep apnea symptoms occurring in the user.

In addition, The snoring and sleep apnea preventer of claim 1, wherein the sensor part transmits the sensing information sensing at least one of lung capacity, body temperature, noise and vibration of the user and oxygen saturation, to the control part.

Further, the control part determines a snoring and sleep apnea state of the user based on oxygen saturation among the sensing information transmitted by the sensor part, controls the operation part in stages according to a preset routine.

Further, wherein the control part includes a 1st measuring member for measuring at least one of lung capacity, body temperature, noise, and vibration of the sensing information transmitted by the sensor part to generate the 1st information: a 2nd measuring member for measuring the oxygen saturation of the sensed information to generate the 2nd information; and a 3rd measuring member for measuring the elapsed time from the point when the sensor unit mounted in the user, and matching the elapsed time with the 1st information and the 2nd information to generate the 3rd information.

And a snoring and sleep apnea prevention system to prevent snoring and sleep apnea from occurring during a user's sleep, comprises, a snoring and sleep apnea preventer; a charging device wherein the preventer is positioned to provide power to the preventer, and to receive information about the snoring and sleep apnea occurring while the preventer mounted in the user: and a management server receiving the information about the snoring and sleep apnea that occurred while the preventer mounted in the user via the charging device, determining the information about the snoring and sleep apnea that occurred while the preventer mounted in the user, and communicating a modified preset routine based on the determined information to the preventer via the charging device.

In addition, the management server receives the 3rd information from the preventer via the charging device, modifies the preset routine of the preventer based on the received 3rd information.

Further, the management server analyzes a pattern of snoring and sleep apnea symptoms of the user based on the 3rd information, and modifies a preset routine based on the analyzed pattern and communicates it to the preventer via the charging device.

Further, the management server determines the degree of snoring and sleep apnea of the user based on matching the elapsed time of the 3rd information with the 2nd information.

Further, wherein the sensor part is formed in the form of a ring to wrap around a part of the user's body, the preventer is mounted on one of the user's two arms.

Further, wherein the sensor part is mounted on at least one of the user's fingers.

Further, wherein the 1st measuring member, the 2nd measuring member and the 3rd measuring member generate different information each other, if a plurality of the sensor parts are provided.

### Advantageous Effects

The snoring and sleep apnea prevention system using the snoring and sleep apnea preventer of the present invention to solve the above problems may have the effect of preventing snoring and sleep apnea symptoms of a user by measuring at least one of lung volume, body temperature, noise and vibration, and oxygen saturation through a sensor part to determine the snoring and sleep apnea symptoms occurring during sleep, and forming a preset routine based on the results.

The effects of the present invention are not limited to the aforementioned effect, and other effects, which are not mentioned above, will be apparent to a person having ordinary skill in the art from the description of the claims.

### DESCRIPTION OF DRAWINGS

A detailed description of according to a preferred embodiment of the present application, which is described below and a summary described above will be able to be more well appreciated when reading the detailed description and the summary in association with the accompanying drawings.

Preferred embodiments are illustrated in the drawings for the purpose of illustrating the present invention.

However, it should be appreciated that the present application is not limited to the illustrated accurate arrangement and means.

FIG. 1 is a diagram illustrating of a snoring and sleep apnea prevention system using a snoring and sleep apnea preventer according to an embodiment of the present invention for a comprehensive explanation;

FIG. 2 is a diagram illustrating a snoring and sleep apnea prevention system using a snoring and sleep apnea preventer in accordance with an embodiment of the present invention;

FIG. 3 is a drawing illustrating an operating part of a snoring and sleep apnea prevention system using a snoring and sleep apnea preventer according to an embodiment of the present invention;

FIG. 4 is a diagram illustrating the overall flow of a snoring and sleep apnea prevention system using a snoring and sleep apnea preventer according to an embodiment of the present invention;

FIG. 5 is a drawing illustrating a charging device for a snoring and sleep apnea prevention system using a snoring and sleep apnea preventer according to an embodiment of the present invention;

FIG. 6 is a drawing illustrating a sensor portion of a snoring and sleep apnea prevention system using a snoring and sleep apnea preventer according to an embodiment of the present invention;

FIG. 7 is a drawing illustrating a sensor portion of a snoring and sleep apnea prevention system using a snoring and sleep apnea preventer according to an embodiment of the present invention;

FIG. 8 is a drawing illustrating a sensor portion of a snoring and sleep apnea prevention system using a snoring and sleep apnea preventer, according to another embodiment of the present invention;

FIG. 9 is a diagram illustrating the flow of sensing information in a snoring and sleep apnea prevention system using a snoring and sleep apnea preventer according to an embodiment of the present invention;

FIG. 10 is a diagram illustrating a management server of a snoring and sleep apnea prevention system using a snoring and sleep apnea preventer according to an embodiment of the present invention;

FIG. 11 is a graph illustrating the relationship between oxygen saturation and AHI in a snoring and sleep apnea prevention system using a snoring and sleep apnea preventer according to an embodiment of the present invention; and

FIG. 12 is a graph illustrating a clearer comparison of the oxygen saturation and AHI of a snoring and sleep apnea prevention system using a snoring and sleep apnea preventer according to an embodiment of the present invention.

### DESCRIPTION OF THE SYMBOL

100: preventer
120: sensor part
122: 1st measuring member
124: 2nd measuring member
126: 3rd measuring member
128: anti-drop member
140: operating part
142: mounting members
144: moving members
146: body member
148: connecting member
160: control part
180: scheduler part
200: charging devices
220: panel part
240: inset part
260: location part
300: management server
320: transmitter/receiver part
340: determinent part
360: routine creation part
380: record part

### Mode for Invention

Hereinafter, a preferred embodiment of the present invention, in which a purpose of the present invention can be realized in detail will be described with reference to the accompanying drawings. In describing the embodiment, the same name and the same reference numeral are used with respect to the same component and the resulting additional description will be omitted.

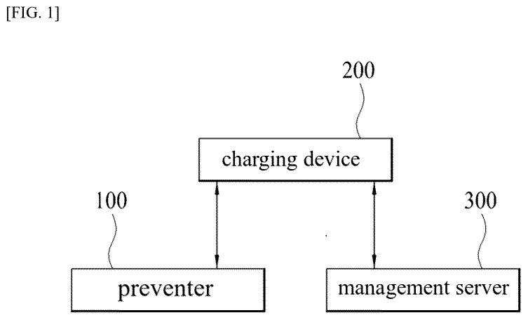

Referring to FIG. 1, a description of a snoring and sleep apnea prevention system using a snoring and sleep apnea preventer according to an embodiment of the present invention can be provided.

FIG. 1 is a diagram illustrating of a snoring and sleep apnea prevention system using a snoring and sleep apnea preventer according to an embodiment of the present invention for a comprehensive explanation.

As shown in FIG. 1, a snoring and sleep apnea prevention system using a snoring and sleep apnea preventer according to an embodiment of the present invention may comprise a preventer 100 for preventing snoring and sleep apnea conditions, charging devices 200 for charging the preventer 100, and a management server 300 for determining and preventing snoring and sleep apnea conditions of a user by receiving information via the charging devices 200.

Wherein the preventer 100 is mounted on a part of a body of the user to prevent snoring and sleep apnea occurring during sleep of the user, and includes a sensor part 120 that collects sensing information to determine the symptoms of snoring and sleep apnea occurring in the user, an operating part 140 mounted in the mouth of the user to move the mandible of the user in accordance with the sensing information, and a control part 160 for controlling the operating part 140 in stages to control the operating part 140 based on the sensing information sensed through the sensor part 120 to control the snoring and sleep apnea symptoms occurring in the user.

Here, the sensor portion 120 may communicate to the control part 160 the sensing information sensing at least one of lung capacity, body temperature, noise and vibration of the user, and blood oxygen saturation.

In this case, the sensing information sensed by the sensor part 120 may be one of the sensing information, as the lung capacity may be measured to determine a response time for controlling the mandible of the user in case the user is in a state of sleep apnea, and the body temperature may affect the number of repetitions or intensity of snoring and sleep apnea depending on the body temperature.

In addition, noise and vibration may be suitable for use as sensing information in snoring and sleep apnea because noise and vibration are measured when snoring occurs, but may be measured in non-snoring situations, and may not be measured when sleep apnea occurs.

In addition, in the case of the blood oxygen saturation of the user, if the sleep apnea persists or the snoring persists, the blood oxygen saturation becomes low due to the lack of oxygen, and accordingly, it is possible to determine that the user has snoring and sleep apnea symptoms.

However, even if the above sensing information is grasped in real time as such, it may be practically difficult to control the mandible of the the user in real time because the quality of sleep of the the user may be lowered due to frequent mandibular movements caused by errors due to the combination of multiple information.

Therefore, as will be described in more detail hereinafter, the sensor portion 120 may collect the sensing information about snoring and sleep apnea occurring while the user is sleeping and may be operated according to a routine established by the control part 160 based on the sensing information.

In particular, although it may vary depending on environmental factors, in general, the position in which a person sleeps, the number or severity of snoring and sleep apnea episodes that occur during sleep, and the cycles of REM sleep in the sleep state, etc. are almost continuously in a similar state from sleep to sleep, so it may be advantageous to identify them and set up a preset routine to ensure that the system is effectively applied to the user.

Meanwhile, the charging devices 200 is formed in a space where the preventive device 100 can be positioned, provides power to the preventive device 100 to charge the preventive device 100, and can receive the sensing information sensed via the sensor part 120 while the user is sleeping, and transmit the sensing information via the control part 160 to the management server 300.

In other words, the preventer 100 is not directly connected to the management server 300 to transmit the sensing information, but may transmit the sensing information to the management server 300 via the charging devices 200 in transmitting the sensing information to the management server 300.

Furthermore, the charging devices 200 may receive the sensing information from the control part 160 to prevent memory overload caused by accumulation of the sensing information by the preventer 100, and may transmit the sensing information received while deleting the sensing information accumulated by the control part 160 to the management server 300, and the management server 300 may set the preset routine based on the sensing information received from the charging devices 200.

At this time, the sensing information received from the management server 300 is continuously accumulated to continuously update the preset routine according to the user's pattern, and the newly updated preset routine can be delivered to the preventer 100 via the charging devices 200.

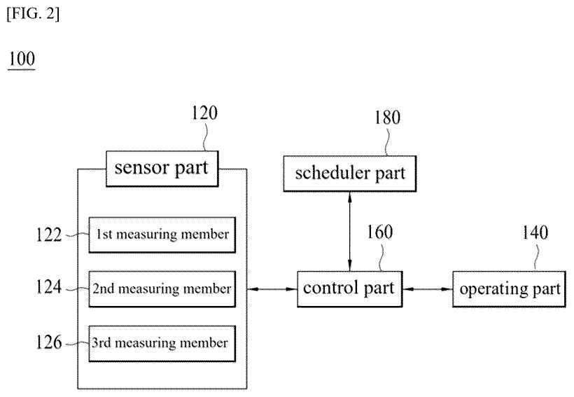

To further illustrate the effectiveness and application of the present invention based on the above-described configuration, reference may be made to FIG. 2 through FIG. 10.

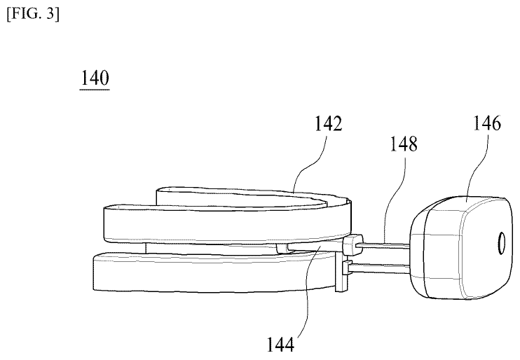

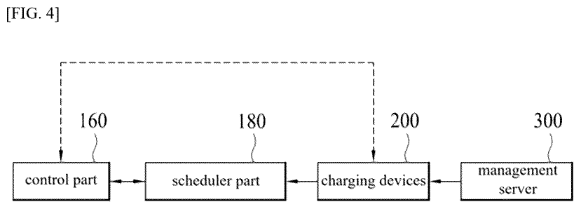

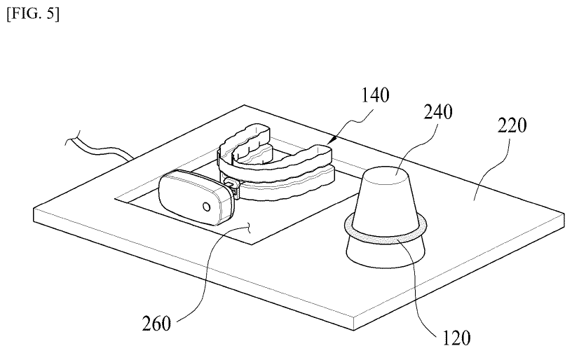

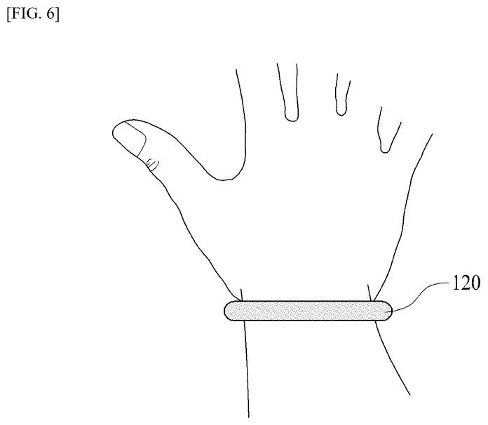

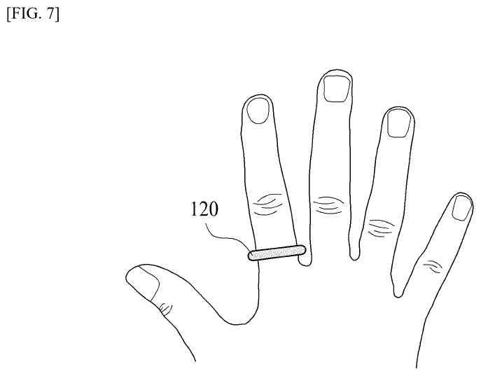

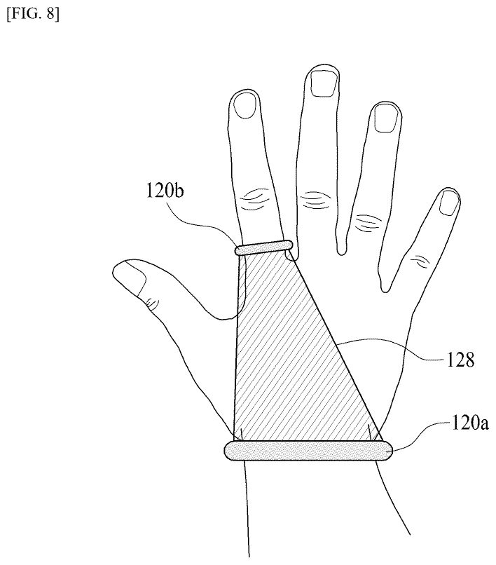

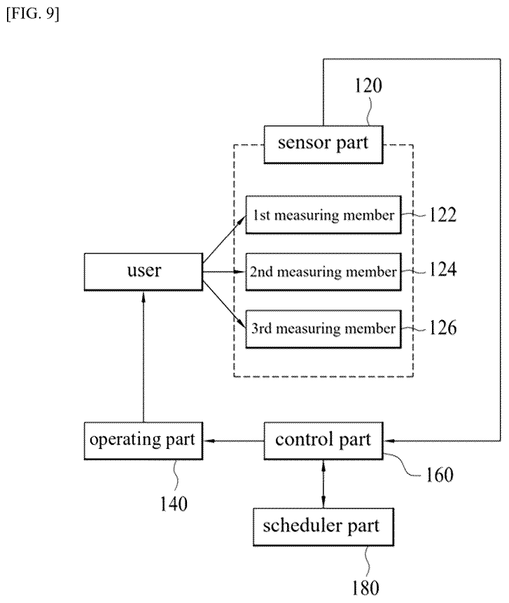

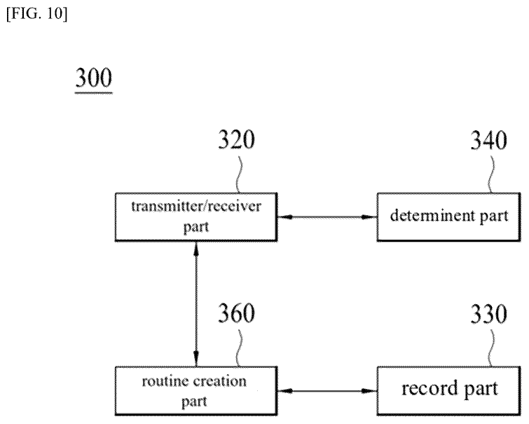

FIG. 2 is a diagram illustrating a snoring and sleep apnea prevention system using a snoring and sleep apnea preventer in accordance with an embodiment of the present invention: FIG. 3 is a drawing illustrating an operating part of a snoring and sleep apnea prevention system using a snoring and sleep apnea preventer according to an embodiment of the present invention: FIG. 4 is a diagram illustrating the overall flow of a snoring and sleep apnea prevention system using a snoring and sleep apnea preventer according to an embodiment of the present invention: FIG. 5 is a drawing illustrating a charging device for a snoring and sleep apnea prevention system using a snoring and sleep apnea preventer according to an embodiment of the present invention: FIG. 6 is a drawing illustrating a sensor portion of a snoring and sleep apnea prevention system using a snoring and sleep apnea preventer according to an embodiment of the present invention: FIG. 7 is a drawing illustrating a sensor portion of a snoring and sleep apnea prevention system using a snoring and sleep apnea preventer according to an embodiment of the present invention: FIG. 8 is a drawing illustrating a sensor portion of a snoring and sleep apnea prevention system using a snoring and sleep apnea preventer, according to another embodiment of the present invention: FIG. 9 is a diagram illustrating the flow of sensing information in a snoring and sleep apnea prevention system using a snoring and sleep apnea preventer according to an embodiment of the present invention: FIG. 10 is a diagram illustrating a management server of a snoring and sleep apnea prevention system using a snoring and sleep apnea preventer according to an embodiment of the present invention.

As shown in FIG. 2, the preventive device 100 of the present invention may further comprise, in addition to the sensor part 120, the operation part 140, and the control part 160 described above, a scheduler part 180 that delivers a preset routine received from the management server 300 to the control part 160 so that the control part 160 can operate the operation part 140 according to the preset routine.

Here, the sensor part 120 may comprise a 1st measuring member 122 that measures at least one of lung capacity, body temperature, noise, and vibration among the sensing information to generate first information, a 2nd measuring member 124 that measures oxygen saturation to generate 2nd information, and a 3rd measuring member 126 that measures the elapsed time from the time the user wears the sensor part 120 to generate 3rd information matching the first information and the 2nd information with the elapsed time.

At this time, the 1st measuring member 122, which measures the first information, delivers the measured sensing information to the control part 160, which can determine from the first information whether the user is in a sleep state or not, and can deliver the accumulated first information to the management server 300 to identify a pattern of the user.

Furthermore, the 2nd measuring member 124 measuring the 2nd information may communicate to the control part 160 a determination that the user is in a snoring and sleep apnea state when oxygen saturation outside a preset value is measured, and may cause the management server 300 to determine the extent of the user's snoring and sleep apnea symptoms based on the accumulated 2nd information.

Furthermore, the 3rd measuring member 126 measuring the 3rd information may generate the 3rd information by matching the first information and the 2nd information measured with the elapsed time to generate the 3rd information, which may enable the management server 300 to determine a general routine that is repeated while the user is sleeping, and to create and modify a preset routine based on the 3rd information by determining a sleep pattern that the user takes while sleeping.

Meanwhile, the first information, the 2nd information, and the 3rd information generated by the 1st measuring member 122, the 2nd measuring member 124, and the 3rd measuring member 126 are communicated to the control part 160, which may share the first information or the 3rd information with the scheduler part 180, and may drive the operation part 140 according to a preset routine communicated by the scheduler part 180.

Furthermore, since the control part 160 prevents the user from snoring and sleep apnea symptoms based on the sensing information measured through the sensor part 120, rather than controlling the operating part 140 in real time, the control part 160 controls the operating part 140 according to a preset routine stored in the scheduler part 180, the preset routine, which is continuously modified according to the accumulation of the sensing information, may have a more suitable effect on the user.

Meanwhile, the operating part 140 is mounted in the mouth of the user and is capable of moving the mandible of the user according to a preset routine controlled by the control part 160.

In this case, the operating part 140 may move the mandible of the user according to a preset routine, but may move the mandible in a stepwise manner so as not to disturb the sleep of the user, which may have the advantage of maintaining the sleep state of the user and preventing the quality of sleep from deteriorating.

Furthermore, as shown in FIG. 3, the operating part 140 may comprise a pair of mounting members 142 that are mounted on the teeth of the user, a moving members 144 that is positioned between the pair of mounting members 142 to move the mandible of the user, a body member 146 in which power is stored to provide power for the moving members 144 to move, power for the control part 160, and a connecting member 148 that connects the moving members 144 to the body member 146.

In this case, the operating part 140 may take the form shown in FIG. 3 to move the mandible of the user, but may take other forms to move the mandible of the user in various ways, and is not necessarily limited thereto.

On the other hand, to briefly summarize the foregoing with reference to FIG. 4, as shown in FIG. 4, the control part 160 may store the information transmitted from the sensor unit 120 and share it with the scheduler part 180, and may transmit the sensing information accumulated overnight from the control part 160 to the charging devices 200.

At this time, the charging devices 200 communicates the sensing information received to the management server 300, and the management server 300 may modify the preset routine based on the sensing information received and communicate it to the charging devices 200.

In this way, the preset routine communicated to the charging devices 200 is communicated to the scheduler part 180 rather than to the control part 160, and the charging devices 200 may cause the sensing information accumulated in the control part 160 to be deleted while communicating the preset routine to the scheduler part 180.

This may be to free up memory for the control part 160 as described above.

Referring to FIG. 5 to illustrate this visually, the charging devices 200, as shown in FIG. 5, may comprise a panel part 220 in which the preventer 100 is positioned, a inset part 240 projecting upwardly from the panel part 220 into which the sensor portion 120 is inserted, and a location part 260 in which the operating part 140 is positioned on the panel part 220 to form a space for providing power to the operating part 140.

Here, the panel part 220 may be formed into an oblong shape such that the sensor portion 120 and the operating part 140 are positioned thereon, and may provide power to the sensor portion 120 and the operating part 140 in a wireless charging method for charging wirelessly.

Furthermore, the inset part 240 may be formed on the panel part 220 in plurality in correspondence with the sensor portion 120 when the sensor portion 120 is provided in plurality, and may extend in a narrowing diameter upwardly from the panel part 220 to prevent the sensor portion 120 from contacting the panel part 220 when the sensor portion 120 is located at any of the upward and downward elevations of the inset part 240.

This may be to prevent the sensor portion 120 from being inserted into the inset part 240, but coming into contact with the panel part 220, making it difficult for the user to subsequently remove the sensor portion 120.

Furthermore, the location part 260 may be formed on the panel part 220, but may be formed as a recessed space in the panel part 220, and may be formed in a form corresponding to the operating part 140, or may be formed to form an oblong space as shown in FIG. 5.

To illustrate various examples of the sensor portion 120 of the present invention, the sensor portion 120 may be conFIG.d in a form that grips the wrist of the user as shown in FIG. 6, and the sensor portion 120 may take the form of a closed loop, or may be formed in a form in which the first end and the other end are freely detachable and bonded.

In other words, it can be a closed loop that fits around the user's wrist, or it can be a ring that fits around the wrist.

In some embodiments, the sensor portion 120 may be provided in a plurality of sensor portions, or may be provided in a form that wraps around one of the user's fingers to prevent discomfort from being caused by the fit of the sensor portion 120 during sleep, and such a form may be preferably in the form of a closed loop that fits around the finger to prevent it from being removed from the user's finger later during sleep.

However, if a plurality of them are provided on the wrist and the finger of the user, the first sensor part 120 located on the wrist and the 2nd sensor part 120 located on the finger may be further provided with an anti-drop member 128 that connects the first sensor part 120 on the wrist and the 2nd sensor part 120 on the finger to each other while preventing the first sensor part 120 and the 2nd sensor part 120 from detaching from the wrist and the finger.

Here, the anti-drop member 128 may comprise an elastic and stretchable material, which may maintain the position of the first sensor part 120 and the 2nd sensor part 120 even while the user is rolling over, which may have the effect of preventing the first sensor part 120 and the 2nd sensor part 120 from being dropped by the anti-drop member 128, since the movement of the 2nd sensor part 120 is restricted by the first sensor part 120.

To summarize and explain this as a whole, as shown in FIG. 9, the user may sleep with the sensor portion 120, the 1st measuring member 122 may generate the first information by sensing at least one of lung capacity, temperature, vibration, and noise as described above, the 2nd measuring member 124 may generate the 2nd information by measuring oxygen saturation of the user, and the 3rd measuring member 126 may generate the 3rd information by matching the first information and the 2nd information with elapsed time.

Upon generating the first information, the 2nd information, and the 3rd information in this manner, the information from the sensor part 120 is communicated to the control part 160, and the control part 160 may share the information communicated from the sensor part 120 with the scheduler part 180.

Furthermore, the control part 160 may receive a preset routine recorded in the scheduler part 180, and may adjust the operating part 140 according to the received preset routine, and the operating part 140 may be operated according to the preset routine of the control part 160, but in a stepwise manner so as not to disturb the sleep of the user.

On the other hand, while the operation part 140 is operated according to the preset routine as described above, it may continuously accumulate data to the management server 300 in order to improve its suitability for the user, the management server 300 includes a transmitter/receiver part 320 that transmits and receives data to the charging devices 200 as shown in FIG. 10, a determinent part 340 that determines the degree of snoring and sleep apnea of the user based on the first information and the 2nd information among the information conveyed by the ttransmitter/receiver part 320, a determinent part 340 that determines the degree of severity of the condition, a routine creation part 360 that modifies a preset routine for controlling the operation part 140 based on the 3rd information, and a record part 380 that is stored and provided to the routine creation part 360 so that the routine creation part 360 can compare the existing preset routine, the newly preset routine, and the pattern of snoring and sleep apnea according to the 3rd information with each other.

Here, the determinent part 340 can be utilized to determine the severity of the symptoms of snoring and sleep apnea of the user as described above, which can be utilized at a later visit to a medical institution to make an accurate diagnosis for alleviating the symptoms of the user, and the routine creation part 360 and the record part 380 can be utilized more effectively, as they can gradually provide customized services to the user according to a more accurate pattern as the number of times the user wears the sensor part 120 increases, thus enabling more effective symptom alleviation.

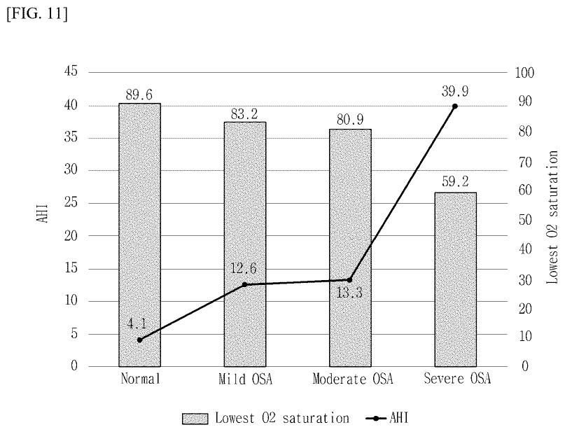

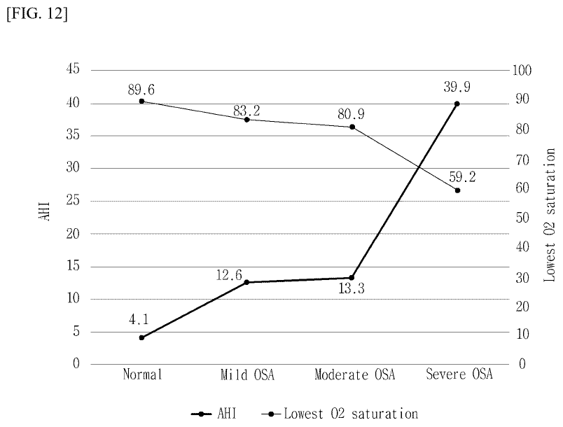

Reference may now be made to FIGS. 11 and 12 to illustrate the suitability of the 2nd measuring member 124 to measure the blood oxygen saturation of the user.

FIG. 11 is shown to illustrate the relationship between oxygen saturation and AHI in a snoring and sleep apnea prevention system using a snoring and sleep apnea preventer according to an embodiment of the present invention, and FIG. 12 is shown to more clearly compare oxygen saturation and AHI in a snoring and sleep apnea prevention system using a snoring and sleep apnea preventer according to an embodiment of the present invention.

As shown in FIG. 11, we can see that most of the time during a normal sleep state, the oxygen saturation is 89.6%, and the AHI, or the number of snoring and sleep apnea repetitions, is relatively low at 4.1.

However, even a 6% drop to 83.2% from a normal sleep state is enough to more than triple the AHI to 12.6, which can be a significant problem, as 12.6 is about every five minutes of snoring and sleep apnea.

Furthermore, it can be seen that even at 80.9%, which is 3% lower than this, the AHI is 13.3, which is roughly 85% of the time, which causes extreme fluctuations in the AHI, and at a level of 13.3, it may be desirable to prevent snoring and sleep apnea by controlling the mandible according to a preset routine, such as the present invention.

Referring to FIG. 12 for contrast, the AHI is shown to increase exponentially with decreasing oxygen saturation, and it may be appropriate to use 85% as a threshold to determine that treatment is required.

Therefore, considering the relationship between AHI and oxygen saturation, acquiring the above 2nd information using the 2nd measuring member 124 can be the to be quite important for alleviating and treating the snoring and sleep apnea symptoms of the above user, and the above 3rd information matched with the elapsed time can also be used to determine how the changes in the AHI and oxygen saturation of the above user have changed, so that the snoring and sleep apnea symptoms of the above user can be alleviated and treated more effectively, and the information can also be utilized when visiting a medical institution.

A preferred embodiment of the present invention has been described as above and a fact that the present invention can be materialized in other specific forms without departing from the gist or scope in addition to the above described embodiment is apparent to those skilled in the art. Therefore, the aforementioned embodiment is not limited but should be considered to be illustrative, and as a result, the present invention is not limited to the above description and may be modified within the scope of the appended claims and a range equivalent thereto.

1. A snoring and sleep apnea preventer to prevent snoring and sleep apnea from occurring during a user's sleep, comprising:
A sensor part mounted on a body part of the user and collecting sensing information to determine snoring and sleep apnea symptoms occurring in the user;
an operating part mounted in the user's mouth to move the user's mandible in response to the sensing information; and
a control part for controlling the operating part based on the sensing information sensed via the sensor part, and controlling the operating part in stages to control snoring and sleep apnea symptoms occurring in the user.

2. The snoring and sleep apnea preventer of claim 1, wherein the sensor part transmits the sensing information sensing at least one of lung capacity, body temperature, noise and vibration of the user and oxygen saturation, to the control part.

3. The snoring and sleep apnea preventer of claim 2, wherein the control part determines a snoring and sleep apnea state of the user based on oxygen saturation among the sensing information transmitted by the sensor part, controls the operation part in stages according to a preset routine.

4. The snoring and sleep apnea preventer of claim 3, wherein the control part determines a snoring and sleep apnea state of the user if the oxygen saturation is within 80 to 90 among the sensing information transmitted by the sensor part.

5. The snoring and sleep apnea preventer of claim 4, wherein the control part includes,
a 1st measuring member for measuring at least one of lung capacity, body temperature, noise, and vibration of the sensing information transmitted by the sensor part to generate the 1st information;
a 2nd measuring member for measuring the oxygen saturation of the sensed information to generate the 2nd information; and
a 3rd measuring member for measuring the elapsed time from the point when the sensor unit mounted in the user, and matching the elapsed time with the 1st information and the 2nd information to generate the 3rd information.

6. A snoring and sleep apnea prevention system to prevent snoring and sleep apnea from occurring during a user's sleep, comprising:
a snoring and sleep apnea preventer of claim 1;
a charging device wherein the preventer is positioned to provide power to the preventer, and to receive information about the snoring and sleep apnea occurring while the preventer mounted in the user; and
a management server receiving the information about the snoring and sleep apnea that occurred while the preventer mounted in the user via the charging device, determining the information about the snoring and sleep apnea that occurred while the preventer mounted in the user, and communicating a modified preset routine based on the determined information to the preventer via the charging device.

7. The snoring and sleep apnea prevention system of claim 6, wherein the management server receives the 3rd information from the preventer via the charging device, modifies the preset routine of the preventer based on the received 3rd information.

8. The snoring and sleep apnea prevention system of claim 7, wherein the management server analyzes a pattern of snoring and sleep apnea symptoms of the user based on the 3rd information, and modifies a preset routine based on the analyzed pattern and communicates it to the preventer via the charging device.

9. The snoring and sleep apnea prevention system of claim 7, wherein the management server determines the degree of snoring and sleep apnea of the user based on matching the elapsed time of the 3rd information with the 2nd information.

10. The snoring and sleep apnea prevention system of claim 6, wherein the sensor part is formed in the form of a ring to wrap around a part of the user's body, the preventer is mounted on one of the user's two arms.

11. The snoring and sleep apnea prevention system of claim 10, wherein the sensor part is mounted on at least one of the user's fingers.

12. The snoring and sleep apnea prevention system of claim 11, wherein the 1st measuring member, the 2nd measuring member and the 3rd measuring member generate different information each other, if a plurality of the sensor parts are provided.
